(19) United States
(12) Patent Application Publication
(10) Pub. No.: US 2013/0079322 A1
(43) Pub. Date: Mar. 28, 2013

(54) CRYSTALLINE OF CARBAPENEM DERIVATIVE OR ITS HYDRATE, PREPARATION METHODS AND USES THEREOF

(71) Applicants: Zhenhua Huang, Jinan City (CN); Yanyan Dong, Jinan City (CN)

(72) Inventors: Zhenhua Huang, Jinan City (CN); Yanyan Dong, Jinan City (CN)

(73) Assignee: Xuanzhu Pharama Co., Ltd., Jinan City (CN)

(21) Appl. No.: 13/699,550

(22) PCT Filed: May 20, 2011
(86) PCT No.: PCT/CN11/00874

§ 371 Date: Dec. 4, 2012

(30) Foreign Application Priority Data
May 21, 2010 (CN) 201010178970.2
Apr. 25, 2011 (CN) 201110104082.0

(51) Int. Cl. C07D 477/20

(52) U.S. Cl. CPC C07D 477/20
USPC 514/210.13; 540/205

(57) ABSTRACT

The present invention relates to a crystalline form of carbapenems derivative (4R,5S,6S)-6-((R)-1-hydroxyethyl)-4-methyl-7-oxo-3-(((3S,5S)-5-((4-sulfamoylbenzyl)carbamoyl)pyrrolidin-3-yl)thio)-1-azabicyclo[3.2.0]hept-2-ene-2-carboxylic acid_as represented by formula (I) or hydrate thereof and the preparation methods thereof, wherein said method comprise: dissolving the compound as represented by formula (I) by an aqueous solution of N,N′-dimethylformamide (DMF), dimethyl sulfoxide (DMSO), and then adding a poor solvent dropwise to this solution, filtering and drying to obtain a crystal. Another method comprises: formulating the compound as represented by formula (I) as an aqueous suspension; after adjusting pH until complete dissolution, adding a mixed solvent of organic solvent/water with a certain volume ratio; adjusting pH to 5.4-7.0, cooling to low temperature, filtering and drying to obtain a crystal. The invention also relates to the use of the crystalline form of compound A or hydrate thereof in the preparation of a medicament for treating and/or preventing infectious diseases. The invention further relates to a pharmaceutical composition comprising the crystalline form of compound A or hydrate thereof and one or more pharmaceutical carriers and/or diluents.

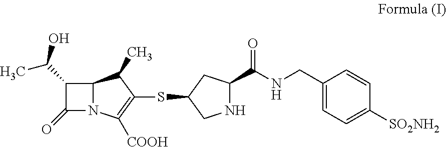 [SMILES: C[C@@H](O)[C@H]1C(=O)N2C(C(=O)O)=C(S[C@@H]3CN[C@H](C(=O)NCc4ccc(S(N)(=O)=O)cc4)C3)[C@H](C)[C@H]12]

### TECHNICAL FIELD

The present invention relates to the field of medical technology, and specifically relates to a crystalline form of the carbapenems derivative (4R,5S,6S)-3-[(3S,5S)-5-[(4-aminosulfonylphen-1-ylmethyl)carbamoyl]-3-pyrrolidinyl]thio-6-[(R)-1-hydroxyethyl]-4-methyl-7-oxo-1-azabicyclo[3.2.0]hept-2-ene-2-carboxylic acid or hydrate thereof and preparation methods thereof, and use thereof in the preparation of a medicament for treating and/or preventing infectious diseases as well as a pharmaceutical composition comprising such compound and one or more pharmaceutical carriers and/or diluents.

### BACKGROUND

Carbapenems are novel β-lactam antibiotics that are developed initially from 1970s of the 20^{th} century. Carbapenems are becoming more and more predominant in clinical use due to its extremely broad spectrum, superiorly high potency, resistance to enzymes and the like.

Currently, the carbapenem antibiotics available on the market are imipenem-cilastatin, panipenem-betamipron, meropenem, ertapenem sodium, biapenem and doripenem. However, the current sorts of carbapenem antibiotics are not ideal: some sorts are unstable to renal dehydropeptidase (DPH-I); some sorts possess central nervous system toxicities; some sorts possess activities against Pseudomonas aeruginosa that are not strong enough, and some sorts possess very low antibacterial activity against Methicillin-resistant Staphylococcus aureus (MRSA). The carbapenem antibiotics as clinically used are all injections, which have very short half life in vivo (except for ertapenem), as well as the shortcomings including fast excretion, high administration frequency in case of serious symptoms, inconvenient clinical administration and the like. Furthermore, along with the global overuse of antibiotics, more and more resistant bacteria have emerged and the resistance of antibiotics is getting worse. Thus, there is a strong need to develop carbapenem antibiotics with preferable antibacterial activity and favorable chemical stability.

The carbapenem derivative (4R,5S,6S)-3-[(3S,5S)-5-[(4-aminosulfonylphen-1-ylmethyl)carbamoyl]-3-pyrrolidinyl]thio-6-[(R)-1 -hydroxyethyl]-4-methyl-7-oxo-1-azabicyclo [3.2.0]hept-2-ene-2-carboxylic acid as represented by formula (I) (refers to compound A for short hereinafter in the specification) produces bactericidal action mainly by binding with penicillin binding protein (PBPs) in the bacterial cell membrane and inhibiting the synthesis of bacterial cell wall, which has preferable bactericidal action against both Gram-positive and Gram-negative bacteria.

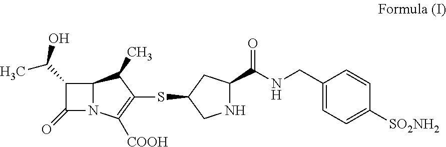 [SMILES: C[C@@H](O)[C@H]1C(=O)N2C(C(=O)O)=C(S[C@@H]3CN[C@H](C(=O)NCc4ccc(S(N)(=O)=O)cc4)C3)[C@H](C)[C@H]12]

The compound A as represented by formula (I) has been described in CN200810127480.2 in detail.

The study on crystalline form is very important in the pharmaceutical development since the different forms of a compound have divergent solubility, and different crystalline forms have a great influence on the stability, operation performance and solubility of a compound. Accordingly, the present inventors have conducted studies on various crystalline forms of compound A and thus indentified the crystalline form of compound A.

### SUMMARY OF THE INVENTION

The object of the invention is to solve the problems as mentioned above by providing a novel crystalline form of compound A with superior stability during preservation, operability and solubility, as well as preparation methods and use thereof.

One object of the invention is to provide the crystalline form of compound A as represented by formula (I) or hydrate thereof, i.e. the crystalline form of carbapenem derivative (4R,5S,6S)-3-[(3S,5S)-5-[(4-aminosulfonylphen-1-yl)methyl]carbamoyl]-3-pyrrolidinyl]thio-6-[(R)-1-hydroxyethyl]-4-methyl-7-oxo-1-azabicyclo [3.2.0]hept-2-ene-2-carboxylic acid as represented by formula (I) or hydrate thereof, characterized in that the X-ray powder diffraction pattern thereof using Cu-Ka radiation represented as 2θ has characteristic peaks at 10.3±0.2, 14.5±0.2, 18.0±0.2, 20.8±0.2, and 23.3±0.2,

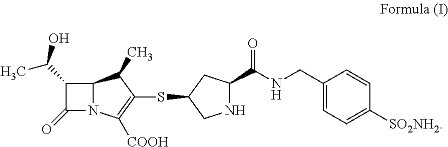 [SMILES: C[C@@H](O)[C@H]1C(=O)N2C(C(=O)O)=C(S[C@@H]3CN[C@H](C(=O)NCc4ccc(S(N)(=O)=O)cc4)C3)[C@H](C)[C@H]12]

The X-ray powder diffraction pattern using Cu-Ka radiation represented as 2θ of said crystalline form of compound A or hydrate thereof has further characteristic peaks at 16.3±0.2, 17.1±0.2, 21.3±0.2, and 22.0±0.2.

The DSC of said crystalline form of compound A or hydrate thereof has the first endothermic transition peak at 56-64° C., and has the second endothermic transition peak at 115-122° C.

The water content of said crystalline form of compound A hydrate is 2%-10%, preferably 5%-10%.

Another object of the present invention is to provide preparation methods of the crystalline form of compound A or hydrate thereof. Compound A can be synthesized by previously known methods, such as the methods disclosed in CN200810127480.2. The crystalline form of compound A (refers to the crystalline form I for short hereinafter) can be obtained by three methods below:

Preparation Method 1:

Dissolving compound A by an aqueous solution of N,N′-dimethylformamide (DMF) or dimethyl sulfoxide (DMSO), and then adding a poor solvent dropwise to this solution, filtering and drying to obtain a crystal.

Said poor solvent is a solvent in which the compound A has a poor solubility, and is selected from the group consisting of lower alcohols containing 1-4 carbon atoms, lower ketones containing 1-6 carbon atoms, acetonitrile, propionitrile, dichloromethane, trichloromethane, nitromethane, diethyl ether, methyl t-butyl ether, anisole, ethyl acetate, ethyl formate, dimethyl carbonate, or tetrahydrofuran, preferably nitromethane or dichloromethane. Said lower alcohols containing 1-4 carbon atoms are selected from the group consisting of methanol, ethanol, propanol, and the like, preferably methanol. Said lower ketones containing 1-6 carbon atoms are selected from the group consisting of acetone, butanone, and the like.

Preparation Method 2:

Formulating compound A as an aqueous suspension; after adjusting pH until complete dissolution, adding a mixed solvent of organic solvent/water with a certain volume ratio; adjusting pH to 5.4-7.0, cooling to low temperature, filtering and drying to obtain a crystal.

Preparation Method 3:

Formulating compound A as an aqueous suspension; after adjusting pH until complete dissolution, absorbing and enriching the solution by column chromatography, then eluting by a mixed solvent of organic solvent/water as an eluant, and distilling off a small part of organic solvent under reduced pressure; concentrating the eluant until a mixed solvent of organic solvent/water with a certain volume ratio is obtained; adjusting pH to 5.4-7.0, cooling to low temperature, filtering and drying to obtain a crystal.

Said pH adjustment is adjusting pH with acids, bases or basic solutions. If pH is adjusted with acids prior to the addition of organic solvent, pH is adjusted with bases or basic solutions after the addition of organic solvent; if pH is adjusted with bases or basic solutions prior to the addition of organic solvent, pH is adjusted with acids after the addition of organic solvent.

In the preparation method 3, the ratio of organic solvent to water in said mixed solvent of organic solvent/water as eluant is 1:0.2˜1:4, preferably 1:0.5˜1:2, most preferably 1:1.

Said mixed solvent of organic solvent/water with a certain volume ratio in the preparation method 2, or mixed solvent of organic solvent/water with a certain volume ratio as obtained after concentration in the preparation method 3 refers to a mixed solvent of acetonitrile/water at a ratio of 1:9˜3:2, or a mixed solvent of methanol/water at a ratio of 1:4˜4:1.

Said acids are inorganic or organic acids, wherein the inorganic acids are selected from the group consisting of hydrobromic acid, hydrochloric acid, sulphuric acid, sulfurous acid, nitric acid or phosphoric acid; the organic acids are selected from the group consisting of methanesulfonic acid, dodecylsulfonic acid, 2-naphthalenesulfonic acid, benzenesulfonic acid, oxalic acid, 2,2-dichloroacetic acid, glycerophosphoric acid, 2-hydroxyethanesulfonic acid, L-aspartic acid, maleic acid, ethanesulfonic acid, 1,5-naphthalenedisulfonic acid, 1,2-ethanedisulfonic acid, cyclohexylaminosulfonic acid, or p-toluenesulfonic acid.

Said bases are organic or inorganic bases, and said basic solutions are solutions as formulated by dissolving organic or inorganic bases in water, wherein the inorganic bases are selected from the group consisting of potassium hydroxide, sodium hydroxide, zinc hydroxide, calcium hydroxide, potassium carbonate, potassium bicarbonate, sodium carbonate, or sodium bicarbonate; the organic bases are selected from the group consisting of L-arginine, betaine, choline, diethylamine, lysine, N,N′-dibenzylethylenediamine, 2-(diethylamino)ethanol, 2-aminoethanol, 1-(2-hydroxyethyl)pyrrole, diethanolamine, dimethylethanolamine, N-methylglucamine, tromethamine, triethanolamine, 4-(2-hydroxyethyl)morpholine, imidazole, or ethanediamine.

Said column chromatography is reverse phase column chromatography, and is selected from the group consisting of C₄ column chromatography, C₈ column chromatography, C₁₈ column chromatography, or resin column chromatography.

Said cooling to low temperature refers to cooling to 0˜10° C.

The present inventors have conducted a number of studies on the preparation methods of the crystalline form I of compound A. The studies shows that after adjusting pH of the aqueous suspension of compound A until complete dissolution, absorbing and enriching by column chromatography, then eluting by a mixed solvent of organic solvent/water; after eluting, concentrating the eluent by distillation under reduced pressure; after distilling off part of the eluant, the ratio of organic solvent to water in the residual eluant is very essential. If the content of organic solvent is lower, the crystalline form II of compound A will be obtained. If the content of organic solvent is higher, the crystalline form I of compound A will be obtained.

In the practical operation “concentrating the eluent by distillation under reduced pressure until a mixed solvent of solvent/water with a certain volume ratio is obtained”, whether the mixed solvent is acetonitrile/water or methanol/water, acetonitrile and methanol are firstly distilled off when distilling the mixed solvent since the boiling points of acetonitrile and methanol are both lower than that of water. After distilling off part of the organic solvent, the residual organic solvent and water have an azeotropic point, and may potentially be distilled off together.

After distilling off part of the eluant under reduced pressure, the ratio of organic solvent to water is difficult to determine quantitatively. Accordingly, the present inventors carried out a series of simulation and verification assays which simulate the ratio of organic solvent to water in the eluant after concentration in order to determine the ratio of organic solvent to water in the eluant after concentration at which the crystalline form II (the crystalline form II of compound A has been described in CN201010190636.9 in detail) and I can be obtained respectively. When the mixed solvent is selected from acetonitrile/water and the ratio of acetonitrile to water is 60:40, by adjusting pH to 5.4-7.0, cooling to low temperature, filtering and drying, a crystal is obtained. If the ratio of acetonitrile to water is higher than 60:40, compound A will not be precipitated. Thus, the upper limit of the ratio of acetonitrile to water is 60:40, i.e. 3:2. After carrying out a number of assays based on dilution with such ratio, the present inventors conclude that when the ratio of acetonitrile to water is no less than 1:9, the crystalline form as obtained finally is the crystalline form I; when the ratio of acetonitrile to water in the eluent is less than 1:9, the crystalline form as obtained finally is the crystalline form II.

When the mixed solvent is selected from methanol/water and the ratio of methanol to water is 80:20, by adjusting pH to 5.4-7.0, cooling to low temperature, filtering and drying, a crystal is obtained. If the ratio of methanol to water is higher than 80:20, compound A will not be precipitated. Thus, the upper limit of the ratio of methanol to water is 80:20, i.e. 4:1. After carrying out a number of assays based on dilution with such ratio, the present inventors conclude that when the ratio of methanol to water is no less than 1:4, the crystalline form as obtained finally is the crystalline form I; when the ratio of methanol to water in the eluent is less than 1:4, the crystalline form as obtained finally is the crystalline form II.

Therefore, the present inventors have determined the ratio of the mixed solvent for obtaining the crystalline form I of compound A, which is 1:9˜3:2 for the mixed solvent of acetonitrile/water, or 1:4˜4:1 for the mixed solvent of methanol/water.

In other words, when column chromatography is used, concentrating the eluent by distillation under reduced pressure until a mixed solvent of organic solvent/water with a certain volume ratio is obtained, at this time, the volume ratio of acetonitrile/water in the mixed solvent after concentration is 1:9˜3:2, or the ratio of methanol/water in the mixed solvent after concentration is 1:4˜4:1, and the crystalline form I of compound A can be obtained at both conditions. When column chromatography is not used, said “a mixed solvent of organic solvent/water with a certain volume ratio” refers to a mixed solvent of acetonitrile/water at a ratio of 1:9˜3:2, or a mixed solvent of methanol/water at a ratio of 1:4˜4:1, and the crystalline form I of compound A can be obtained at both conditions.

The advantages of the methods for preparing the crystalline form I of compound A of the present invention lie in the following aspects: the organic solvents as used in the methods of the present invention are commonly used organic solvents in pharmaceutical field, which possess lower boiling points, can be easily removed and hardly remain; the operability for technical amplification is excellent and the cost is lower.

The crystalline form of compound A or hydrate thereof as obtained by the above methods (refers to the crystalline form I for short hereinafter) is assayed by:

(1) Powder X-Ray Diffraction

Condition for X-ray diffraction assay: Cu-Ka ray, 1.54 Å (monochromator), measured by D/MAX-RB X-ray diffractometer.

The X-ray powder diffraction pattern using Cu-Ka radiation represented as 2θ has strong characteristic peaks at 10.3±0.2, 14.5±0.2, 18.0±0.2, 20.8±0.2, and 23.3±0.2; and has further characteristic peaks at 16.3±0.2, 17.1±0.2, 21.3±0.2, and 22.0±0.2.

When the X-ray diffraction is used for measuring the crystalline form of the present invention, errors should be taken into account as determining the structure of the crystalline form, since the apparatus or condition used for measurement may result in little measurement errors to the peaks measured. Accordingly, the present inventors have considered the error range (±0.2) when determining the 2θ angles.

(2) DSC Endothermic Assay

Condition for the DSC assay: DZ3335 differential scanning calorimeter, protected by nitrogen gas, at a heating rate of 5° C./min.

The DSC has the first endothermic transition peak at 56-64° C., and has the second endothermic transition peak at 115-122° C.

(3) Water Content Assay

Measured by the Karl Fischer Water Determination method (refers to the K-F method for short), the crystalline form I of compound A of the present invention has a water content of 2%-10%, preferably 5%-10%. Accordingly, the crystalline form I of compound A of the present invention can be in the form of hydrate.

The present invention further provides the use of the crystalline form of compound A or hydrate thereof in the preparation of a medicament for treating and/or preventing infectious diseases.

The present invention also provides a pharmaceutical composition comprising the crystalline form of compound A or hydrate thereof and one or more pharmaceutical carriers and/or diluents, which can be any one of pharmaceutically acceptable dosage forms, such as injections.

### BRIEF DESCRIPTION OF THE DRAWINGS

FIG. 1 is the X-ray powder diffraction pattern of the crystalline form I of compound A as represented by formula (I). The ordinate represents diffraction intensity (CPS), and the abscissa represents diffraction angle (2θ).

FIG. 2 is the DSC pattern of the crystalline form I of compound A as represented by formula (I). The ordinate represents power (mW), and the abscissa represents temperature (° C.).

### EXAMPLES

The present invention is further illustrated by the examples in detail hereinafter. The examples are merely illustrative and should not be construed as limitations upon the scope of the present invention. The technical solutions and variants thereof as obtained based on the above disclosure of the invention fall into the scope of the present invention entirely.

### Preparative Example

### Preparation of Amorphous Compound A

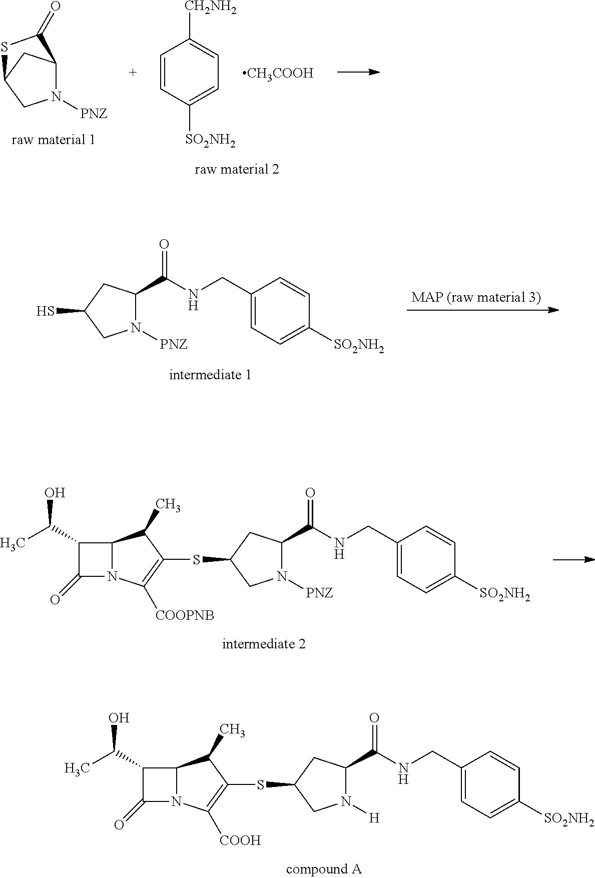

In the above synthetic route, —PNZ represents

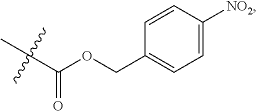 [SMILES: *C(=O)OCc1ccc([N+](=O)[O-])cc1]

—PNB represents

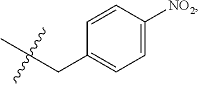 [SMILES: *Cc1ccc([N+](=O)[O-])cc1]

MAP represents

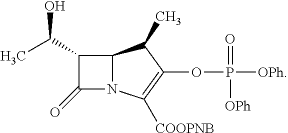

(1) preparative example of (2S,4S)-2-[(4-aminosulfonylphen-1-yl)iminomethylformyl]-4-thiol-1-(p-nitrobenzyloxycarbonyl)pyrrolidine (intermediate 1)

5-N-(4-nitrobenzyloxycarbonyl)-2-thia-5-azabicyclo[2.2.1]hept-3 -one (raw material 1) (1600 g, 5.19 mol) and mafenide acetate (raw material 2) (1219.2 g, 4.95 mol) were dissolved in acetonitrile, and the solution was warmed to 40° C. Triethylamine was added dropwise under nitrogen protection, and the reaction mixture was stirred to precipitate, filtered to obtain intermediate 1.

(2) preparative example of (4R,5S,6S)-3-[[(3S,5S)-N-(4-nitrobenzyloxycarbonyl)-5-[(4-aminosulfonylphen-1-yl)methyl]carbamoyl]-3-pyrrolidinyl]thio-6-[(R)-1-hydroxyethyl]-4-methyl-7-oxo-1-azabicyclo[3.2.0]hept-2-ene-2-carboxylic acid(4-nitrobenzyl) methyl ester (intermediate 2)

Intermediate 1 and [(4R,5S,6S)-3-[(diphenoxyphosphono)oxy]-6-[(R)-1-hydroxyethyl]-4-methyl-7-oxo-1-azabicyclo[3.2.0]hept-2-ene-2-carboxylic acid(4-nitrobenzyl) methyl ester (raw material 3) (2980 g, 5.01 mol) were dissolved in dimethylformamide, and the solution was cooled to −15° C. Triethylamine was added dropwise under nitrogen protection. After complete reaction, ethyl acetate was added. The mixture was sequentially washed with water, dilute hydrochloric acid and saturated sodium bicarbonate, and the organic layer was dried over anhydrous sodium sulfate. The ethyl acetate layer was concentrated and refluxed to precipitate, filtered, and dried to obtain 3300 g of solid (i.e. intermediate 2) with a yield of 79.46% and a purity of 94.75%.

(3) preparative example of amorphous (4R,5S,6S)-3-[(3S,5S)-5-[(4-aminosulfonylphen-1-yl)methyl]carbamoyl]-3-pyrrolidinyl]thio-6-[(R)-1-hydroxyethyl]-4-methyl-7-oxo-1-azabicyclo [3.2.0]hept-2-ene-2-carboxylic acid (amorphous compound A)

Intermediate 2 (500 g, 0.60 mol) was dissolved in tetrahydrofuran, and sodium bicarbonate (100 g, 1.19 mol) as well as 100.0 g of 10% anhydrous palladium on carbon were added into water. The two solutions were placed into hydrogenation reactor and mixed. The hydrogenation reactor was filled with hydrogen gas, pressurized to 4.0 MPa, warmed to 30° C., and stirred until complete reaction. After filtration, the reaction mixture was washed with ethyl acetate. The pH of water layer was adjusted with acetic acid to 6. Subsequently, the mixture was separated by column chromatography with octadecyl bonded silica gel, purified by preparative liquid chromatography, lyophilizated to obtain 145.8 g of solid (i.e. amorphous compound A) with a yield of 46.32% and a purity of 96.66%.

Molecular formula: C₂₂H₂₈N₄O₇S₂

Molecular weight: 524.6

¹H-NMR(D₂O, 600 MHz): δ1.05 (d, 3H), 1.15 (d, 3H), 1.97 (m, 1H), 2.83 (m, 1H), 3.20 (m, 1H), 3.30 (m, 1H), 3.33 (m, 1H), 3.63 (dd, 1H), 3.92 (m, 1H), 4.08 (m, 1H), 4.10 (m, 1H), 4.33 (d, 1H), 4.40 (dd, 1H), 4.45 (d, 1H), 7.40 (d, 2H), 7.78(d, 2H).

### Example 1

### Preparation 1 of the Crystalline Form I of Compound A

600 mg of compound A was dissolved with 2 mL of water and 3 mL of dimethyl sulfoxide (DMSO), and 50 mL of nitromethane was added dropwise with stirring. The mixture was stirred for 0.5-1 h at room temperature, filtered, dried under vacuum to obtain 300 mg of white crystal.

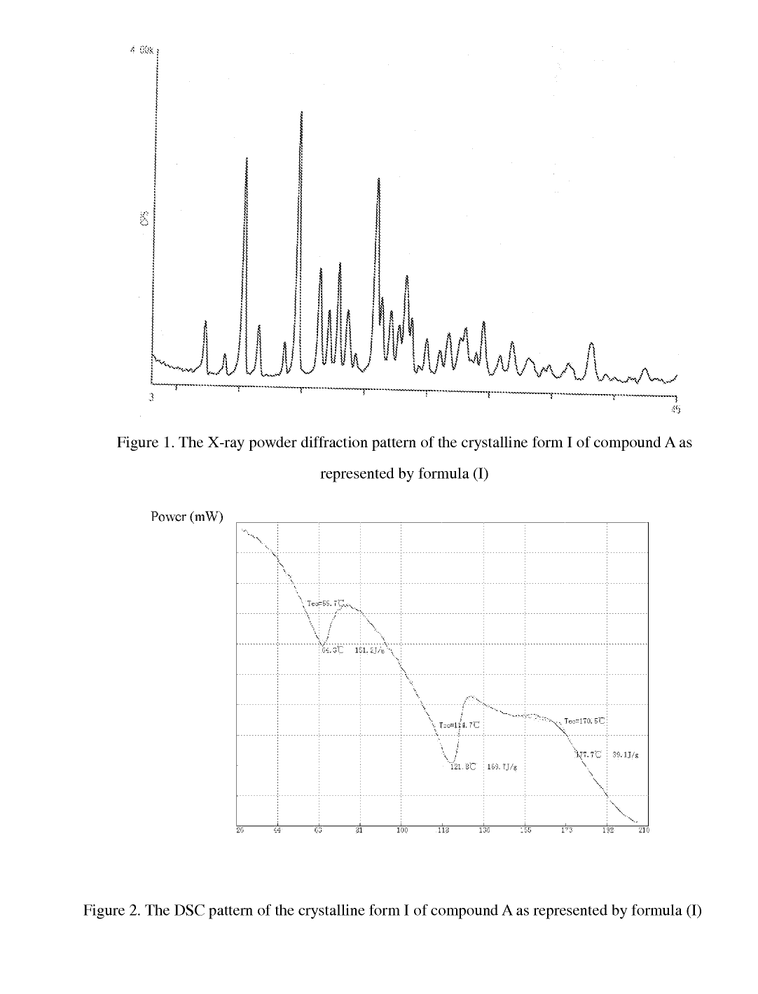

XRD diffraction: the results of XRD diffraction assay are shown in FIG. 1.

Water content (the K-F method): 2.44%.

### Example 2

### Preparation 2 of the Crystalline Form I of Compound A

Referring to the procedure of example 1, dimethylsulfoxide (DMSO) was replaced by N,N′-dimethylformamide (DMF), nitromethane was replaced by methanol, and 320 mg of white crystal was obtained.

XRD diffraction: the diffraction angle (2θ) shows the characteristic peaks at the following positions in the XRD diffraction pattern: 10.24, 14.52, 16.30, 17.08, 17.84, 20.70, 21.28, 21.94, and 23.14.

Water content (the K-F method): 2.81%.

### Example 3

### Preparation 3 of the Crystalline Form I of Compound A

Referring to the procedure of example 1, dimethyl sulfoxide (DMSO) was replaced by N,N′-dimethylformamide (DMF), nitromethane was replaced by dichloromethane, and 380 mg of white crystal was obtained.

XRD diffraction: the diffraction angle (2θ) shows characteristic peaks at the following positions in the XRD diffraction pattern: 10.28, 14.56, 16.34, 17.12, 17.88, 20.80, 21.30, 22.02, and 23.24.

Water content (the K-F method): 5.54%.

### Example 4

### Preparation 4 of the Crystalline Form I of Compound A

400 mL of deionized water was added to 7.0 g of compound A, and the resultant mixture was stirred to form a suspension. Then the suspension was adjusted pH with 3.36 g of sodium bicarbonate solid until complete dissolution. Afterwards, the solution was enriched with C₁₈ column chromatography, and then eluted with a mixed solution of acetonitrile/water at a ratio of 1:1. The eluent was concentrated by distillation under reduced pressure to ⅔ of the original volume, adjusted with 2N hydrochloric acid to pH 6, left to rest at 0-5° C. to obtain a crystal, which was filtered, washed with cold water, and dried under vacuum to obtain 5.26 g of the crystalline form I of compound A with a yield of 75.1%.

XRD diffraction: the crystal has characteristic peaks at the following diffraction angles (2θ) in the XRD diffraction pattern: 10.40, 14.70, 16.50, 17.24, 18.02, 20.94, 21.42, 22.18, and 23.40.

### Example 5

### Preparation 5 of the Crystalline Form I of Compound A

250 mL of deionized water was added to 3.96 g of compound A, and the resultant mixture was stirred to form a suspension. Then the suspension was adjusted pH with 2.7 g of sodium bicarbonate solid until complete dissolution. Afterwards, the solution was enriched with C₁₈ column chromatography, and then eluted with a mixed solution of methanol/water at a ratio of 1:1. The eluent was concentrated by distillation under reduced pressure to ⅔ of the original volume, adjusted with 2N hydrochloric acid to pH 6.50 mg of crystal seed of crystalline form I was added to this solution. The mixture was left to rest at 0-5° C. to obtain a crystal, which was filtered, washed with cold water, and dried under vacuum to obtain 2.40 g of the crystalline form I of compound A with a yield of 60.6%.

XRD diffraction: the crystal has characteristic peaks at the following diffraction angles (2θ) in the XRD diffraction pattern: 10.30, 14.60, 16.40, 17.16, 17.92, 20.86, 21.34, 22.08, and 23.26.

### Example 6

### Preparation 6 of the Crystalline Form I of Compound A

5 mL of deionized water was added to 500 mg of compound A, and the resultant mixture was stirred to form a suspension, which was adjusted pH with 0.25 g of sodium bicarbonate solid until complete dissolution. Afterwards, 550 μL of acetonitrile (volume ratio: acetonitrile/water=1:9) was added. The resultant mixture was adjusted with 2N hydrochloric acid to pH 6, filtered, washed with cold water, and dried under vacuum to obtain 310 mg of the crystalline form I of compound A with a yield of 62.0%.

XRD diffraction: the crystal has characteristic peaks at the following diffraction angles (2θ) in the XRD diffraction pattern: 10.26, 14.56, 16.36, 17.12, 17.86, 20.84, 21.30, 22.04, and 23.28.

### Example 7

### Preparation 7 of the Crystalline Form I of Compound A

20 mL of deionized water was added to 2.0 g of compound A, and the resultant mixture was stirred to form a suspension, which was adjusted pH with 1.25 g of sodium bicarbonate solid until complete dissolution. Afterwards, 5 mL of methanol (volume ratio: methanol/water=1:4) was added. The resultant mixture was adjusted with 2N hydrochloric acid to pH 6, filtered, washed with cold water, and dried under vacuum to obtain 1.33 g of the crystalline form I of compound A with a yield of 66.5%.

### Example 8

### Preparation 8 of the Crystalline Form I of Compound A

The specific method is the same as that of example 6. Volume ratio: acetonitrile/water=15:85.

### Example 9

### Preparation 9 of the Crystalline Form I of Compound A

The specific method is the same as that of example 6. Volume ratio: acetonitrile/water=20:80.

### Example 10

### Preparation 10 of the Crystalline Form I of Compound A

The specific method is the same as that of example 6. Volume ratio: acetonitrile/water=30:70.

### Example 11

### Preparation 11 of the Crystalline Form I of Compound A

The specific method is the same as that of example 6. Volume ratio: acetonitrile/water=40:60.

### Example 12

### Preparation 12 of the Crystalline Form I of Compound A

The specific method is the same as that of example 6. Volume ratio: acetonitrile/water=50:50.

### Example 13

### Preparation 13 of the Crystalline Form I of Compound A

The specific method is the same as that of example 6. Volume ratio: acetonitrile/water=60:40.

### Example 14

### Preparation 14 of the Crystalline Form I of Compound A

The specific method is the same as that of example 7. Volume ratio: methanol/water=30:70.

### Example 15

### Preparation 15 of the Crystalline Form I of Compound A

The specific method is the same as that of example 7. Volume ratio: methanol/water=40:60.

### Example 16

### Preparation 16 of the Crystalline Form I of Compound A

The specific method is the same as that of example 7. Volume ratio: methanol/water=50:50.

### Example 17

### Preparation 17 of the Crystalline Form I of Compound A

The specific method is the same as that of example 7. Volume ratio: methanol/water=60:40.

### Example 18

### Preparation 18 of the Crystalline Form I of Compound A

The specific method is the same as that of example 7. Volume ratio: methanol/water=70:30.

### Example 19

### Preparation 19 of the Crystalline Form I of Compound A

The specific method is the same as that of example 7. Volume ratio: methanol/water=80:20.

### Example 20

### Stability Assay for the Crystalline Form I of Compound A

Condition for the assay: sampled after placing for 5 days, 10 days at a high temperature of 60° C., and the results thus obtained were compared with those obtained at day 0 by measuring related substances and contents thereof. The samples were sealed with plastic bags coated with aluminium foils during the assay.

Test Compound:

Amorphous compound A (refers to the amorphous for short hereinafter): self-prepared. Preparation method: see the preparation of amorphous compound A in the above preparative examples. The purity is 96.66%, and the batch number is 110901.

The crystalline form I of compound A (refers to the crystalline form I for short hereinafter): prepared by example 1 of the Specification. The batch number is 20100315-4.

Content determination is performed by external standard method using the sample at day 0 as control according to the high performance liquid chromatography in Chinese Pharmacopoeia, 2010 edition, appendix VD.

Operation Condition

Apparatus: high performance liquid chromatograph (Agilent 1200 series)

Chromatographic column: Agilent C₁₈; filler: 5 μm octadecyl silane boned silica gel; inner diameter: 4.6 mm; length of column: 150 mm.

Column temperature: 30° C.

Mobile phase: 0.02 M diammonium phosphate (pH=5.2 adjusted by phosphoric acid):acetonitrile=100:7

Flow rate: 1.0 mL/min

Sample size: 10μL

Determination of related substances is performed by area normalization method according to the high performance liquid chromatography in Chinese Pharmacopoeia, 2010 edition, appendix VD.

Operation Condition

Apparatus: high performance liquid chromatograph (Agilent 1200 series)

Chromatographic column: Agilent C₁₈; filler: 5 μm octadecyl silane boned silica gel; inner diameter: 4.6 mm; length of column: 150 mm.

Column temperature: 30° C.

Mobile phase: A: 0.02 M diammonium phosphate (pH=5.2 adjusted by phosphoric acid):acetonitrile=95:5.
B: 0.02 M diammonium phosphate (pH=5.2 adjusted by phosphoric acid):acetonitrile=30:70.

Gradient condition: see Table 1

TABLE 1
Chromatographic gradient condition for
determination of related substances

| T (min) | 0 | 5 | 15 | 18 | 25 | 30 | 35 | 40 | 42 | 47 |
|---|---|---|---|---|---|---|---|---|---|---|
| B % | 0 | 3 | 4 | 15 | 30 | 65 | 100 | 100 | 0 | 0 |

Flow rate: 1.0 mL/min

Sample size: 10 μL

The experimental results obtained: see Table 2

TABLE 2
Results of stability assay

| Sample name | | Related substance % | | | | | Content |
|---|---|---|---|---|---|---|---|
| (batch number) | Day | open ring % | increase_{open ring} % | total % | increaseₜₒₜₐₗ % | Content % | decrease % |
| The crystalline | 0 | 0.136 | — | 1.709 | — | 100.00 | — |
| form I | 5 | 0.782 | 0.646 | 2.550 | 0.841 | 96.27 | 3.73 |
| (20100315-4) | 10 | 1.120 | 0.984 | 2.876 | 1.167 | 96.05 | 3.95 |
| The amorphous | 0 | 0.476 | — | 9.295 | — | 100.00 | — |
| (110901) | 5 | 3.328 | 2.852 | 20.675 | 11.38 | 73.16 | 26.84 |

Note:
compound A is easily hydrolyzed. After hydrolysis, the β-lactam ring is opened, which results in the main related substance briefly called the open ring product, and the content of the open ring product is considered as a factor for stability determination. In table 2, open ring % refers to the content of open ring product; total % refers to the total content of related substances; increase_{open ring} % refers to the increased amount of the open ring product placed for 5 and 10 days at a high temperature of 60° C. as compared with the amount at day 0; increaseₜₒₜₐₗ % refers to the increased amount of total related substances placed for 5 and 10 days at a high temperature of 60° C. as compared with the amount at day 0.

The open ring product:

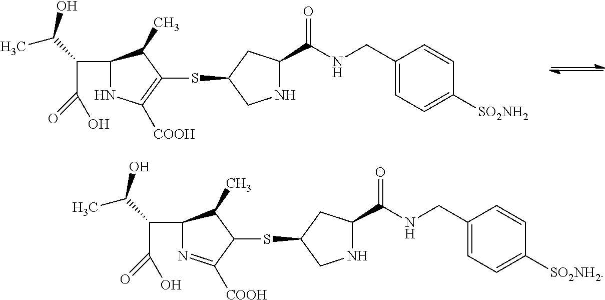

It is shown from table 2 that after placed for 5 and 10 days at a high temperature of 60° C., the content of the crystalline form I and the amorphous of compound A decreased, while the content of the open ring product in the related substances and total substances increased. However, after placed for 5 and 10 days at a high temperature of 60° C., the increased amount of the open ring product of the crystalline form I was lower than that of the amorphous; the increased amount of the content of total related substances of the crystalline form I was lower than that of the amorphous; the decreased amount of the content of the crystalline form I was lower than that of the amorphous. Accordingly, as compared with the amorphous of compound A, the increase in the content of related substances of the crystalline form I of compound A is lower, and the decrease in the content of the crystalline form I is lower, such that the crystalline form I of compound A surpasses the amorphous in each aspect of properties and the properties thereof are more stable.

### Example 21

### Preparative Example of the Injection of the Crystalline Form I of Compound A

1. Formula:

| The crystalline form I of | 250 g (calculated | |
|---|---|---|
| compound A | as C₂₂H₂₈N₄O₇S₂) | |
| Anhydrous sodium | 50 | g |
| carbonate | | |
| Totally prepared | 1000 | bottles |

2. Procedure:

(1) The antibiotic glass bottles and rubber plugs used for the preparation were washed, dried and sterilized;

(2) the air-condition and dehumidification equipment were turned on, controlling the inside relative humidity of the grade 100 laminar flow room within 50%;

(3) the crystalline form I of compound A and anhydrous sodium carbonate were weighed according to the formula, and were uniformly mixed to obtain a sterile mixed powder;

(4) the semifinished products were measured;

(5) separately loaded;

(6) plugs were added;

(7) capped;

(8) the finished products were generally inspected;

(9) packed and warehoused.

### Example 22

### Antibacterial Activity Assay of the Crystalline Form I of Compound A

Test strains: the following clinically isolated strains were provided by public organizations.

Gram-positive bacteria: methicillin sensitive Staphylococcus aureus (MSSA), and penicillin sensitive Streptococcus pneumoniae;

Gram-negative bacteria: ESBLs negative Escherichia coli, ESBLs positive Escherichia coli, Haemophilus influenzae, and Moraxelle catarrhalis.

Test compound: the crystalline form I of compound A

Experimental method: standard agar double dilution method was adopted.

### Experimental Results and Conclusions

### Antibacterial Activity of the Crystalline Form I of Compound A Against Clinically Isolated Strains

| Strains | MIC_{mode}(mg/L) | MIC₉₀ (mg/L) |
|---|---|---|
| MSSA | 0.062 | 0.062 |
| Penicillin sensitive | 0.008 | 0.031 |
| Streptococcus pneumoniae | | |
| ESBLs negative | 0.016 | 0.016 |
| Escherichia coli | | |
| ESBLs positive | 0.016 | 0.25 |
| Escherichia coli | | |
| Haemophilus influenzae | 0.062 | 0.125 |
| Moraxelle catarrhalis | 0.004 | 0.008 |

The above experimental results indicate that the crystalline form I of compound A exhibits excellent antibacterial activity against both Gram-positive and Gram-negative bacteria, and has a broad antibacterial spectrum and a superior potential for clinical application.

The present invention is described and illustrated in detail hereinbefore; however, the scope thereof is not confined to it. All the modifications, amendments, improvements and changes to the technical solutions of the present invention are within the spirit and scope of the present invention as defined by the appended claims.

1. The crystalline form of carbapenem derivative (4R,5S,6S)-6-(R)-1-hydroxyethyl)-4-methyl-7-oxo-3-(((3S,5S)-5-(4-sulfamoylbenzyl)carbamoyl)pyrrolidin-3-yl)thio)-1-azabicyclo[3.2.0]hept-2-ene-2-carboxylic acid as represented by formula (I) or hydrate thereof, characterized in that the X-ray powder diffraction pattern thereof using Cu-Ka radiation represented as 2θ has characteristic peaks at 10.3±0.2, 14.5±0.2, 18.0±0.2, 20.8±0.2, and 23.3±0.2,

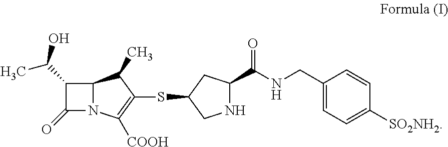 [SMILES: C[C@@H](O)[C@H]1C(=O)N2C(C(=O)O)=C(S[C@@H]3CN[C@H](C(=O)NCc4ccc(S(N)(=O)=O)cc4)C3)[C@H](C)[C@H]12]

2. The crystalline form of claim 1, characterized in that the X-ray powder diffraction pattern using Cu-Ka radiation represented as 2θ has further characteristic peaks at 16.3±0.2, 17.1±0.2, 21.3±0.2, and 22.0±0.2.

3. The crystalline form of claim 1 or 2, characterized in that the DSC thereof has the first endothermic transition peak at 56-64° C., and has the second endothermic transition peak at 115-122° C.

4. The crystalline form of claim 1 or 2, characterized in that the water content thereof is 2%-10%, preferably 5%-10%.

5. A method for preparing the crystalline form of carbapenem derivative (4R,5S,6S)-6-(R)-1-hydroxyethyl)-4-methyl-7-oxo-3-(((3S,5S)-5-(4-sulfamoylbenzyl)carbamoyl)pyrrolidin-3-yl)thio)-1-azabicyclo[3.2.0]hept-2-ene-2-carboxylic acid as represented by formula (I) or hydrate thereof of claim 1 or 2, characterized in that: dissolving the compound as represented by formula (I) by an aqueous solution of N,N′-dimethylformamide (DMF) or dimethyl sulfoxide (DMSO), and then adding a poor solvent dropwise to this solution, filtering and drying to obtain a crystal.

6. The method of claim 5, wherein the poor solvent is a solvent in which the (4R,5S,6S)-6-((R)-1-hydroxyethyl)-4-methyl-7-oxo-3-(((3S,5S)-5-((4-sulfamoylbenzyl)carbamoyl)pyrolidin-3-yl)thio)-1-azabicyclo[3.2.0]hept-2-ene-2-carboxylic acid has a poor solubility, and is selected from the group consisting of lower alcohols containing 1-4 carbon atoms, lower ketones containing 1-6 carbon atoms, acetonitrile, propionitrile, dichloromethane, trichloromethane, nitromethane, diethyl ether, methyl t-butyl ether, anisole, ethyl acetate, ethyl formate, dimethyl carbonate, or tetrahydrofuran, preferably nitromethane or dichloromethane.

7. The method of claim 6, wherein said lower alcohols containing 1-4 carbon atoms are selected from the group consisting of methanol, ethanol, propanol, preferably methanol; said lower ketones containing 1-6 carbon atoms are selected from the group consisting of acetone and butanone.

8. A method for preparing the crystalline form of carbapenem derivative (4R,5S,6S)-6-(R)-1-hydroxyethyl)-4-methyl-7-oxo-3-(((3S,5S)-5-(4-sulfamoylbenzyl)carbamoyl)pyrrolidin-3-yl)thio)-1-azabicyclo[3.2.0]hept-2-ene-2-carboxylic acid as represented by formula (I) or hydrate thereof of claim 1 or 2, characterized in that: formulating the compound as represented by formula (I) as an aqueous suspension; after adjusting pH until complete dissolution, adding a mixed solvent of organic solvent/water with a certain volume ratio; adjusting pH to 5.4-7.0, cooling to low temperature, filtering and drying to obtain a crystal.

9. A method for preparing the crystalline form of carbapenem derivative (4R,5S,6S)-6-(R)-1-hydroxyethyl)-4-methyl-7-oxo-3-(((3S,5S)-5-(4-sulfamoylbenzyl)carbamoyl)pyrrolidin-3-yl)thio)-1-azabicyclo[3.2.0]hept-2-ene-2-carboxylic acid as represented by formula (I) or hydrate thereof of claim 1 or 2, characterized in that: formulating the compound as represented by formula (I) as an aqueous suspension; after adjusting pH until complete dissolution, absorbing and enriching the solution by column chromatography, then eluting by a mixed solvent of organic solvent/water as an eluant, and distilling off a small part of organic solvent under reduced pressure; concentrating the eluant until a mixed solvent of organic solvent/water with a certain volume ratio is obtained; adjusting pH to 5.4-7.0, cooling to low temperature, filtering and drying to obtain a crystal.

10. The method of claim 8, wherein said pH adjustment is adjusting pH with acids, bases or basic solutions, if pH is adjusted with acids prior to the addition of organic solvent, pH is adjusted with bases or basic solutions after the addition of organic solvent; if pH is adjusted with bases or basic solutions prior to the addition of organic solvent, pH is adjusted with acids after the addition of organic solvent.

11. The method of claim 9, wherein the ratio of organic solvent to water in said mixed solvent of organic solvent/water as eluant is 1:0.2˜1:4, preferably 1:0.5˜1:2, most preferably 1:1.

12. The method of claim 8, wherein said mixed solvent of organic solvent/water with a certain volume ratio is a mixed solvent of acetonitrile/water at a ratio of 1:9˜3:2, or a mixed solvent of methanol/water at a ratio of 1:4˜4:1.

13. The method of claim 10, wherein said acids are inorganic or organic acids, wherein the inorganic acids are selected from the group consisting of hydrobromic acid, hydrochloric acid, sulphuric acid, sulfurous acid, nitric acid or phosphoric acid; wherein the organic acids are selected from the group consisting of methanesulfonic acid, dodecylsulfonic acid, 2-naphthalenesulfonic acid, benzenesulfonic acid, oxalic acid, 2,2-dichloroacetic acid, glycerophosphoric acid, 2-hydroxyethanesulfonic acid, L-aspartic acid, maleic acid, ethanesulfonic acid, 1,5-naphthalenedisulfonic acid, ethane-1,2-disulfonic acid, cyclohexylaminosulfonic acid, or p-toluenesulfonic acid.

14. The method of claim 10, wherein said bases are organic or inorganic bases, and said basic solutions are solutions as formulated by dissolving organic or inorganic bases in water; wherein the inorganic bases are selected from the group consisting of potassium hydroxide, sodium hydroxide, zinc hydroxide, calcium hydroxide, potassium carbonate, potassium bicarbonate, sodium carbonate, or sodium bicarbonate; wherein the organic bases are selected from the group consisting of L-arginine, betaine, choline, diethylamine, lysine, N,N′-dibenzylethylenediamine, 2-(diethylamino)ethanol, 2-aminoethanol, 1-(2-hydroxyethyl)pyrrole, diethanolamine, dimethylethanolamine, N-methylglucamine, tromethamine, triethanolamine, 4-(2-hydroxyethyl)morpholine, imidazole, or ethanediamine.

15. The method of claim 9, wherein said column chromatography is reverse phase column chromatography, and is selected from the group consisting of C₄ column chromatography, C₈ column chromatography, C₁₈ column chromatography, or resin column chromatography.

16. The method of claim 8, wherein said cooling to low temperature refers to cooling to 0˜10° C.

17. The use of the crystalline form of claim 1 or 2 in the preparation of a medicament for treating and/or preventing infectious diseases.

18. A pharmaceutical composition comprising the crystalline form of claim 1 or 2 and one or more pharmaceutical carriers and/or diluents, which can be any one of pharmaceutical acceptable dosage forms.

19. The pharmaceutical composition of claim 18, wherein said dosage forms are injections.

20. The method of claim 9, wherein said pH adjustment is adjusting pH with acids, bases or basic solutions, if pH is adjusted with acids prior to the addition of organic solvent, pH is adjusted with bases or basic solutions after the addition of organic solvent; if pH is adjusted with bases or basic solutions prior to the addition of organic solvent, pH is adjusted with acids after the addition of organic solvent.

21. The method of claim 20, wherein said acids are inorganic or organic acids, wherein the inorganic acids are selected from the group consisting of hydrobromic acid, hydrochloric acid, sulphuric acid, sulfurous acid, nitric acid or phosphoric acid; wherein the organic acids are selected from the group consisting of methanesulfonic acid, dodecylsulfonic acid, 2-naphthalenesulfonic acid, benzenesulfonic acid, oxalic acid, 2,2-dichloroacetic acid, glycerophosphoric acid, 2-hydroxyethanesulfonic acid, L-aspartic acid, maleic acid, ethanesulfonic acid, 1,5-naphthalenedisulfonic acid, ethane-1,2-disulfonic acid, cyclohexylaminosulfonic acid, or p-toluenesulfonic acid.

22. The method of claim 20, wherein said bases are organic or inorganic bases, and said basic solutions are solutions as formulated by dissolving organic or inorganic bases in water; wherein the inorganic bases are selected from the group consisting of potassium hydroxide, sodium hydroxide, zinc hydroxide, calcium hydroxide, potassium carbonate, potassium bicarbonate, sodium carbonate, or sodium bicarbonate; wherein the organic bases are selected from the group consisting of L-arginine, betaine, choline, diethylamine, lysine, N,N′-dibenzylethylenediamine, 2-(diethylamino)ethanol, 2-aminoethanol, 1-(2-hydroxyethyl)pyrrole, diethanolamine, dimethylethanolamine, N-methylglucamine, tromethamine, triethanolamine, 4-(2-hydroxyethyl)morpholine, imidazole, or ethanediamine.

23. The method of claim 9, wherein said mixed solvent of organic solvent/water with a certain volume ratio is a mixed solvent of acetonitrile/water at a ratio of 1:9˜3:2, or a mixed solvent of methanol/water at a ratio of 1:4˜4:1.

24. The method of claim 9, wherein said cooling to low temperature refers to cooling to 0˜10° C.
